(19) United States
(12) Patent Application Publication
(10) Pub. No.: US 2022/0122719 A1
(43) Pub. Date: Apr. 21, 2022

(54) SYSTEMS AND METHODS FOR PERFORMING SURGERY

(71) Applicant: Avail Medsystems, Inc., Palo Alto, CA (US)

(72) Inventor: Daniel Hawkins, Palo Alto, CA (US)

(21) Appl. No.: 17/218,687

(22) Filed: Mar. 31, 2021

Related U.S. Application Data
(63) Continuation of application No. 16/723,203, filed on Dec. 20, 2019, now abandoned.
(60) Provisional application No. 62/783,131, filed on Dec. 20, 2018.

(51) Int. Cl. G16H 40/20; G06F 3/01

(52) U.S. Cl. CPC G16H 40/20; G06F 3/01

(57) ABSTRACT

Systems and methods for performing surgery are provided herein. The systems and methods may comprise performing a surgery based on an input provided by a vendor representative that is associated with one or more tools or instruments usable to perform one or more steps of the surgery. The input may comprise surgical information associated with a visual representation that is generated for the surgery and provided to the vendor representative. The surgical information may be associated with the visual representation and the one or more tools or instruments usable to perform the one or more steps of the one or more surgical procedures.

### BACKGROUND OF THE INVENTION

Medical products may be used during medical procedures, and may be associated with vendors that provide the medical products. Traditionally, vendor representatives may be present in-person during the medical procedures where associated products are being used, to provide support. Such in-person support can be time consuming and costly, as it requires that the vendor representative physically make his or her way to the various locations of the related medical procedures.

Furthermore scheduling of operations is often conducted for various health care facilities on an ad hoc basis. Such traditional techniques for scheduling and tracking operations are also cumbersome for determining when and where vendor representatives are needed, and for making schedules for the vendor representatives. Oftentimes, the time of vendor representatives are not efficiently allocated. Furthermore access by medical personnel to vendor representatives may be limited by the physical constraints.

### SUMMARY OF THE INVENTION

A need exists for improved systems and methods of communication. A need exists for systems and methods that allow for vendors to communicate remotely with relevant personnel during a medical procedure. A further need exists for systems that aid in the automation of determining where a given vendor representative's attention is needed and facilitating the communication connection.

Aspects of the invention are directed to a method of communication with a vendor representative, said method comprising: obtaining medical procedure scheduling information for one or more health care facilities; determining, with aid of a processor and based on the medical procedure scheduling information, a vendor representative that may facilitate one or more medical procedures identified in the medical procedure scheduling information; presenting, on a user interface of a device of the vendor representative, a visual representation of a medical procedure at a health care facility that would benefit from the vendor representative at the time that the visual representation is displayed; and creating a communication channel between the device of the vendor representative, and a device of medical personnel at a location for the medical procedure in response to a single interaction between the vendor representative and the visual representation.

Additional aspects and advantages of the present disclosure will become readily apparent to those skilled in this art from the following detailed description, wherein only exemplary embodiments of the present disclosure are shown and described, simply by way of illustration of the best mode contemplated for carrying out the present disclosure. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example of a medical communication system between an operating room and one or more remote individuals, in accordance with embodiments of the invention.

FIG. 2 shows an example of a system where multiple vendor representatives may be capable of communicating with multiple health care facilities, in accordance with embodiments of the invention.

FIG. 3 shows an example of communication between different devices for a given entity, in accordance with embodiments of the invention.

FIG. 4 shows an example of scheduling information for various procedures at a given health care facility, in accordance with embodiments of the invention.

FIG. 5 shows an example of relationships between various representatives, vendors, and an exemplary health care facilities, in accordance with embodiments of the invention.

FIG. 6 shows an example of a user interface presented to a vendor representative, in accordance with embodiments of the invention.

FIG. 7 shows an additional example of a user interface presented to a vendor representative, in accordance with embodiments of the invention.

FIG. 8 shows an example of a user interface where a vendor representative may be able to establish connections with relevant personnel, in accordance with embodiments of the invention.

FIG. 9 shows an example of a user interface when communication has been established, in accordance with embodiments of the invention.

FIG. 10 shows an exemplary computer system, in accordance with embodiments of the invention.

### DETAILED DESCRIPTION OF THE INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

The invention provides systems and methods for communication facilitation. Various aspects of the invention described herein may be applied to any of the particular applications set forth below. The invention may be applied as a part of a health care scheduling system or communication system. It shall be understood that different aspects of the invention can be appreciated individually, collectively or in combination with each other.

A communication system may aid in providing communications between vendor representatives and medical personnel who may be performing a medical procedure. The medical personnel may use a medical product that is supported by a vendor representative. The communication system may allow a vendor to provide a remove telepresence during a medical procedure, and support the medical product as needed.

The communication system may be integrated with a scheduling system for a health care facility. The communication system may optionally interface with a pre-existing scheduling system at a health care facility. The communication system may automatically utilize information from the scheduling system to present relevant options to provide support to vendor representatives. In some embodiments, the communication system may automatically determine one or multiple vendor representatives that may provide support during a particular medical procedure and present a visual representation of the medical procedure. A vendor representative may interact with the visual representation of the medical procedure to be automatically connected to the medical personnel. In some instances, the vendor may be connected after a single, simple interaction with the visual representation.

Any description herein of a vendor representative may apply to any other type of individual that may provide support during a medical procedure. For instance, any description herein of a vendor representative may also apply to outside medical professionals or specialists, consultants, technicians, manufacturers, financial support, social workers, or any other individuals.

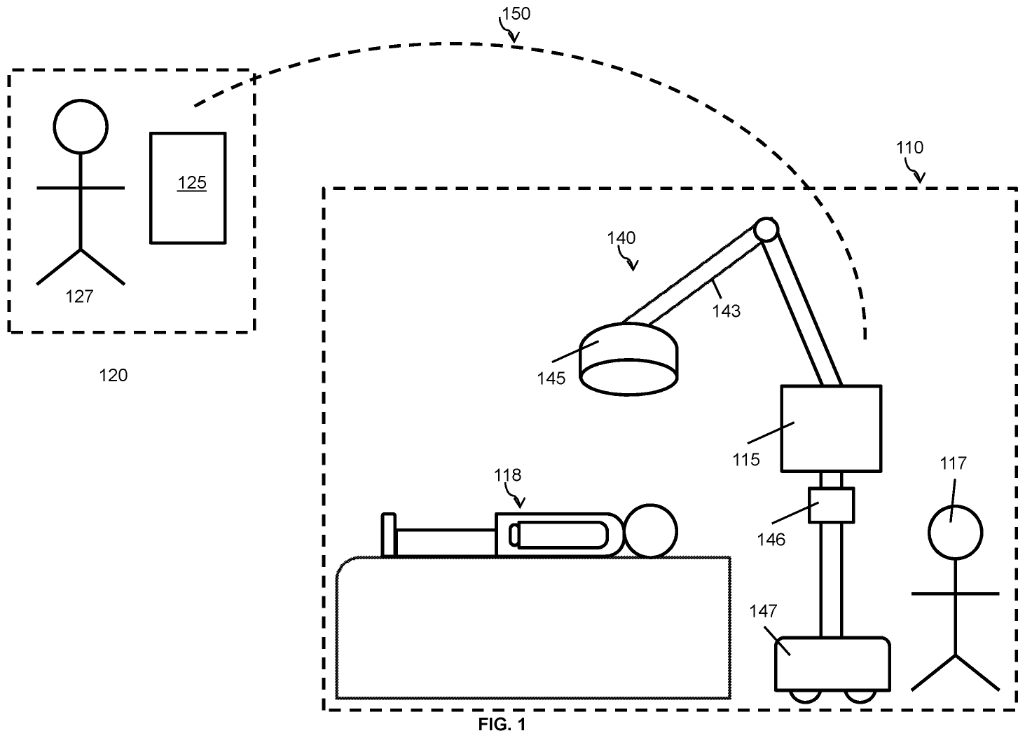

FIG. 1 shows an example of a medical communication system between an operating room and one or more remote individuals, in accordance with embodiments of the invention. Communication may be provided between a first location 110 and a second location 120.

The first location 110 may be an operating room of a health care facility. A health care facility may be any type of facility or organization that may provide some level of health care or assistance. In some examples, health care facilities may include hospitals, clinics, urgent care facilities, out-patient facilities, ambulatory surgical centers, nursing homes, hospice care, home care, rehabilitation centers, laboratory, imaging center, veterinary clinics, or any other types of facility that may provide care or assistance. A health care facility may or may not be provided primarily for short term care, or for long-term care. A health care facility may be open at all days and times, or may have limited hours during which it is open. A health care facility may or may not include specialized equipment to help deliver care. Care may be provided to individuals with chronic or acute conditions. A health care facility may employ the use of one or more health care providers (a.k.a. medical personnel). Any description herein of a health care facility may refer to a hospital or any other type of health care facility, and vice versa.

The first location may be any room or region within a health care facility. For example, the first location may be an operating room, surgical suite, clinic room, triage center, emergency room, or any other location. The first location may be within a region of a room or an entirety of a room. The first location may be any location where an operation may occur, where surgery may take place, where a medical procedure may occur, and/or where a medical product is used. In one example, the first location may be an operating room with a patient 118 that is being operated on, and one or more medical personnel 117, such as a surgeon or surgical assistant that is performing the operation, or aiding in performing the operation. Medical personnel may include any individuals who are performing the medical procedure or aiding in performing the medical procedure. Medical personnel may include individuals who provide support for the medical procedure. For example, the medical personnel may include a surgeon performing a surgery, a nurse, an anesthesiologist, and so forth. Examples of medical personnel may include physicians (e.g., surgeons, anesthesiologists, radiologists, internists, residents, oncologists, hematologists, cardiologists, etc.), nurses (e.g., CNRA, operating room nurse, circulating nurse), physicians' assistants, surgical techs, and so forth. Medical personnel may include individuals who are present for the medical procedure and authorized to be present.

Medical products may include devices that are used alone or in combination with other devices for therapeutic or diagnostic purposes. Medical products may be medical devices. Medical products may include any products that are used during an operation to perform the operation or facilitate the performance of the operation. Medical products may include tools, instruments, implants, prostheses, disposables, or any other apparatus, appliance, software, or materials that may be intended by the manufacturer to be used for human beings. Medical products may be used for diagnosis, monitoring, treatment, alleviation, or compensation for an injury or handicap. Medical products may be used for diagnosis, prevention, monitoring, treatment, or alleviation of disease. In some instances, medical products may be used for investigation, replacement, or modification of anatomy or of a physiological process. Some examples of medical products may range from surgical instruments (e.g., handheld or robotic), catheters, endoscopes, stents, pacemakers, artificial joints, spine stabilizers, disposable gloves, gauze, IV fluids, drugs, and so forth.

Medical products may be provided by one or more vendors. Typically, vendors may make arrangements with health care facilities to provide medical products. Vendors may be entities, such as companies, that manufacture and/or distribute medical products. The vendors may have representatives that may be able to provide support to personnel using the medical devices. The vendor representatives (who may also be known as product specialists or device reps), may be knowledgeable about one or more particular medical products. Vendor representatives may aid medical personnel (e.g., surgeons, surgical assistants, physicians, nurses) with any questions they may have about the medical products. Vendor representatives may aid in selection of sizing or different models of particular medical products. Vendor representatives may aid in function of medical products. Vendor representatives may help a medical personnel use product, or troubleshoot any issues that may arise. These questions may arise in real-time as the medical personnel are using a product. For instance, questions may arise about a medical product while a surgeon is in an operating room to perform a surgery. Traditionally, medical representatives have been located at the first location with the medical personnel. However, this can be time consuming since the medical representative will need to travel to the location of the medical procedure. Secondly, the medical representative may be present but the medical representative's help may not always be needed, or may be needed for a very limited time. Then, the medical representative may have to travel to another location. It may be advantageous for a medical representative to communicate remotely as needed with personnel at the first location.

A second location 120 may be any location where a vendor representative 127 is located. The second location may be remote to the first location. For instance, if the first location is a hospital, the second location may be outside the hospital. In some instances, the first and second locations may be within the same building but in different rooms, floors, or wings. The second location may be at an office of the vendor representative. A second location may be at a residence of a vendor representative.

A vendor representative may have a rep communication device 125 which may communicate with a local communication device 115 at the first location. Any form of communication channel 150 may be formed between the rep communication device and the location communication device. The communication channel may be a direct communication channel or indirect communication channel. The communication channel may employ wired communications, wireless communications, or both. The communications may occur over a network, such as a local area network (LAN), wide area network (WAN) such as the Internet, or any form of telecommunications network (e.g., cellular service network). Communications employed may include, but are not limited to 3G, 4G, LTE communications, and/or Bluetooth, infrared, radio, or other communications. Communications may optionally be aided by routers, satellites, towers, and/or wires. The communications may or may not utilize existing communication networks at the first location and/or second location.

Communications between rep communication devices and local communication devices may be encrypted. Optionally, only authorized and authenticated rep communication devices and local communication devices may be able to communicate over a communication system.

In some embodiments, a rep communication device and/or local communication device may communicate with one another through a communication system. The communication system may facilitate the connection between the rep communication device and the local communication device. The communication system may aid in accessing scheduling information at a health care facility. The communication system may aid in presenting, on a rep communication device, a user interface to a vendor representative about one or more possible medical procedures that may benefit from the vendor representative's support.

A communication device, such as a rep communication device 125 and/or a local communication device 115 may have one or more cameras. The one or more cameras may be integral to the communication device. Alternatively, the one or more cameras may be removable and/or connectable to the communication device. The one or more cameras may face a user when the user looks at a display of the communication device. The one or more cameras may face away from a user when the user looks at a display of the communication device. In some instances, multiple cameras may be provided which may face in different directions. The cameras may be capable of capturing images at a desired resolution. For instance, the cameras may be capable of capturing images at least a 6 mega pixel, 8 mega pixel, 10 mega pixel, 12 mega pixel, 20 mega pixel, 4K, or 8K resolution. A camera on a rep communication device may capture an image of a vendor representative. A camera on a local communication device may capture an image of a medical personnel. A camera on a local communication device may capture an image of a surgical site and/or medical tools, instruments or products.

The communication device may comprise one or more microphones or speakers. A microphone may capture audible noises such as the voice of a user. For instance, the rep communication device microphone may capture the speech of the vendor representative and a local communication device microphone may capture the speech of a medical personnel. One or more speakers may be provided to play sound. For instance, a speaker on a rep communication device may allow a vendor representative to hear sounds captured by a local communication device, and vice versa.

A communication device may comprise a display screen. The display screen may be a touchscreen. The display screen may accept inputs by a user's touch, such as finger. The display screen may accept inputs by a stylus or other tool.

A communication device may be any type of device capable of communication. For instance, a communication device may be a smartphone, tablet, laptop, desktop, server, personal digital assistant, wearable (e.g., smartwatch, glasses, etc.), or any other type of device.

In some embodiments, a local communication device 115 may be supported by a medical console 140. The local communication device may be permanently attached to the medical console, or may be removable from the medical console. In some instances, the local communication device may remain functional while removed from the medical console. The medical console may optionally provide power to the local communication device when the local communication device is attached to (e.g., docked with) the medical console. The medical console may be mobile console that may move from location to location. For instance, the medical console may include wheels that may allow the medical console to be wheeled from location to location. The wheels may be locked into place at desired locations. The medical device may optionally comprise a lower rack and/or support base 147. The lower rack and/or support base may house one or more components, such as communication components, power components, auxiliary inputs, and/or processors.

The medical console may optionally include one or more cameras 145, 146. The cameras may be capable of capturing images of the patient 118, or portion of the patient (e.g., surgical site). The cameras may be capable of capturing images of the medical devices. The cameras may be capable of capturing images of the medical devices as they rest on a tray, or when they are handled by a medical personnel and/or used at the surgical site. The cameras may be capable of capturing images at any resolution, such as those described elsewhere herein. The cameras may be used to capture a still images and/or video images. The cameras may be capturing images in real time.

One or more of the cameras may be movable relative to the medical console. For instance, one or more cameras may be supported by an arm. The arm may include one or more sections. In one example, a camera may be supported at or near an end of an arm. The arm may include one or more sections, two or more section, three or more sections, four or more sections, or more sections. The sections may move relative to one another or a body of the medical console. The sections may pivot about one or more hinge. A camera may have at least one, two, three, or more degrees of freedom. An arm may optionally include a handle that may allow a user to manually manipulate the arm to a desired position. The arm may remain in a position to which it has been manipulated. A user may or may not need to lock an arm to maintain its position. This may provide a steady support for a camera. The arm may be unlocked and/or re-manipulated to new positions as needed.

In some embodiments, one or more cameras may be provided at the second location. The one or more cameras may or may not be supported by the medical console. In some embodiments, one or more cameras may be supported by a ceiling, wall, furniture, or other items at the second location. The one or more cameras may be fixed cameras or movable cameras. The one or more cameras may be capable of rotating about one or more, two or more, or three or more axes. The one or more cameras may include pan-tilt-zoom cameras.

Images captured by the one or more cameras 145, 146 may be sent to a rep communication device. This may allow the vendor representative to remotely view images captured by the field of view of the camera. For instance, the vendor may view the surgical site and/or any medical devices being used. The vendor may be able to view the medical personnel. The vendor may be able to view these in substantially real-time. For instance, this may be within 1 minutes or less, 30 seconds or less, 20 seconds or less, 15 seconds or less, 10 seconds or less, 5 seconds or less, 3 seconds or less, 2 seconds or less, or 1 second or less of an event actually occurring.

This may allow a vendor representative to lend aid or support without needing to be physically at the first location. The medical console and cameras may aid in providing the vendor representative with the necessary images and information to have a virtual presence at the first location.

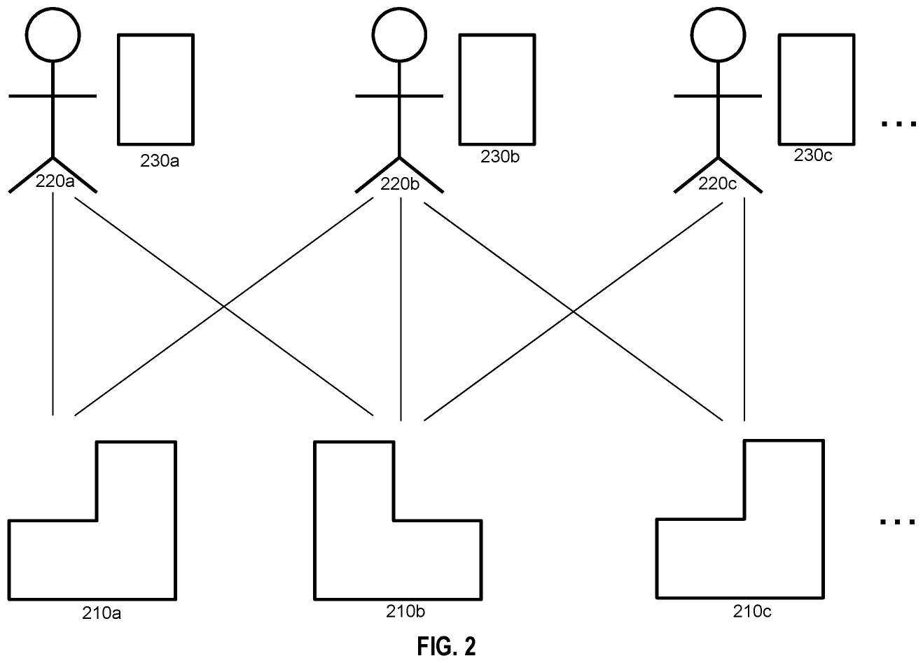

FIG. 2 shows an example of a system where multiple vendor representatives may be capable of communicating with multiple health care facilities, in accordance with embodiments of the invention.

Various health care facilities 210a, 210b, 210c may be provided. The health care facilities may be of the same or different type. For example, the health care facilities may include hospitals.

One or more vendors may provide medical products to the various health care facilities. For instance, a first vendor company may provide one or more types of medical products to a first health facility and a second health care facility. A second vendor company may provide one or more types of medical products to a first health facility and a second health care facility. A vendor company may provide one or more vendor representatives 220a, 220b, 220c that may provide support for various medical products. The vendor representatives may provide support to one or more multiple health care facilities. The vendor representatives may utilize respective rep communication devices 230a, 230b, 230c.

As illustrated, various vendor representatives 220a, 220b, 220c may provide support to various health care facilities 210a, 210b, 210c. Such support may overlap with other vendor representatives, or may not overlap. Such support may overlap for similar products or different products. As illustrated, when vendor representatives are providing support to multiple different health care facilities, allowing a vendor representative to provide support remotely may advantageously save the vendor representative a large amount of time.

To further improve the efficiency and ease for vendor representatives to communicate with the appropriate medical personnel and lend support, the systems and methods are provided herein that facilitate the communication. The health care facilities and the vendors may utilize communication systems and methods as provided herein to utilize the facilitated communication that allows for remote vendor representative support.

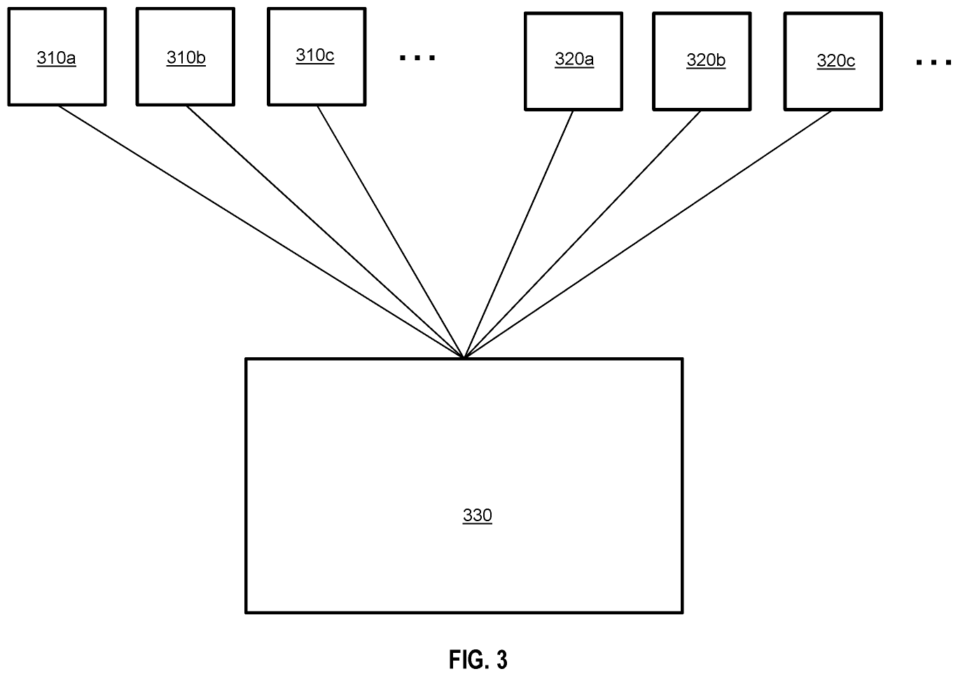

FIG. 3 shows an example of communication between different devices for a given entity, in accordance with embodiments of the invention. As previously described, one or more rep communication devices 310a, 310b, 310c may communicate with one or more local communication devices 320a, 320b, 320c. A rep communication device may be utilized by a vendor representative. A local communication device may be utilized at a site of a medical procedure (e.g., operating room at a hospital), optionally by medical personnel.

An entity 330 may facilitate the communications between the rep communication devices and the local communication devices. The entity may be a third party that is different from the vendors and/or health care facilities. The entity may run or operate a communication system. The communication facilitation entity may optionally provide a platform and/or infrastructure to facilitate the communications. In some instances, the communication facilitation entity may utilize cloud computing infrastructure to provide communication services. The entity may optionally provide software and/or applications that may facilitate the communications. The entity may provide a portal through which the rep communication devices and the vendor communication devices may connect. Tangible, non-transitory computer readable media may be provided comprising code, logic, or instructions that may facilitate the communications between the rep communication devices and the local communication devices.

Communications may be initiated by one or more local communication devices. For instance, the one or more local communication devices may initiate a call or communication to one or more rep communication devices. The rep communication devices may be able to accept, reject, and/or ignore the communication. Additionally and/or alternatively, communications may be initiated by one or more rep communication devices. For instance, the one or more rep communication devices may initiate a call or communication to one or more local communication devices. The local communication devices may be able to accept, reject and/or ignore the communication.

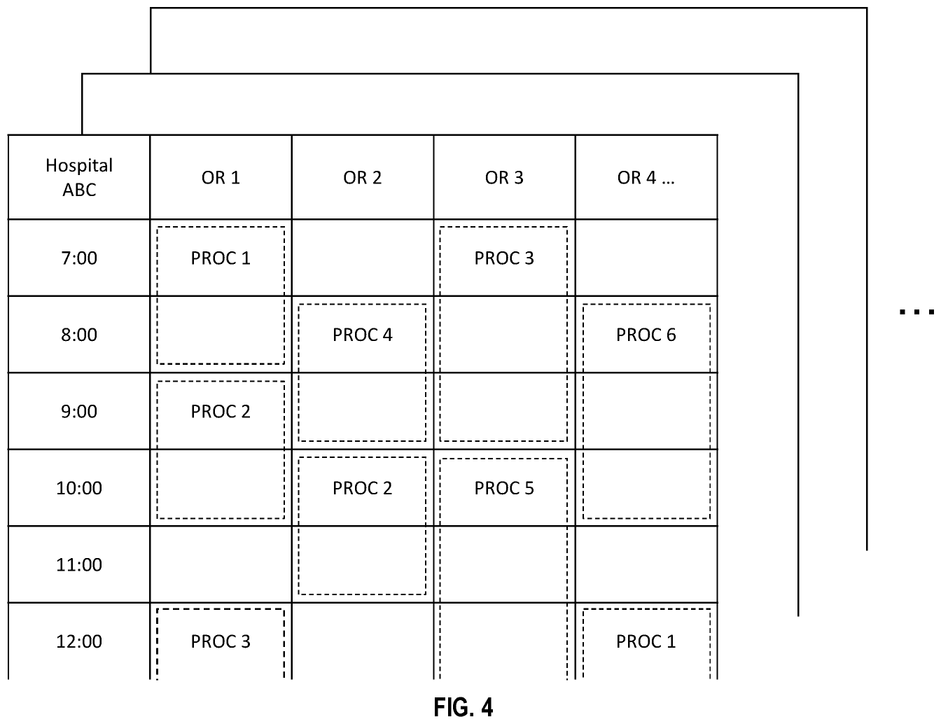

FIG. 4 shows an example of scheduling information for various procedures at a given health care facility, in accordance with embodiments of the invention. The scheduling information for Hospital ABC is provided by way of example only. The scheduling information may be provided for multiple health care facilities. The multiple health care facilities may comprise hospitals and/or other types of health care facilities.

The scheduling information for a particular health care facility, such as Hospital ABC, may include multiple locations where vendor support may be needed. For example, the multiple locations may include multiple operating rooms (e.g., OR1, OR2, OR3, OR4, etc.). Each health facility may have an independent number of locations.

The schedule may also include timing information, such as time of day for a particular day. In some instances, the scheduling information may be updated in real-time. Scheduling information may be available for the current day, upcoming day, the next few days, the next week, the next month, etc. The scheduling information may be updated in real-time, or may be updated periodically (e.g., daily, every several hours, every hour, every 30 minutes, every 15 minutes, every 10 minutes, every 5 minutes, every minute, every second, or more frequently). The scheduling information may be updated in response to an event.

The scheduling information may include information about a procedure that may occur at the various locations. In some instances, the information about the procedure may include a procedure code. The procedure code may be specific to a health care facility or may follow a governmental or industry standard. In some instances, procedure codes may include codes such as ICD-10 procedure coding system (ICD-10-PCS), healthcare common procedure coding system (including current procedural terminology), nursing interventions classifications (NIC), international classification of primary care (ICPC-2), international classification of procedures in medicine (ICPM), international classification of health interventions (ICHI), or other codes.

The scheduling information may include additional information about procedures. For example, Procedure 1 may be scheduled to occur at 7:00 AM in OR1. Procedure 4 may be scheduled to occur at 8:00 AM in OR 2. The procedures may be of different type. For example, the multiple occurs of PROC 1 may indicate procedures of the same type. Indicates of PROC 1 and PROC 2 may indicate that these are procedures of different type. The estimated length of time for each procedure may or may not be provided.

Examples of different types of procedures may include but are not limited to thoracic surgery, orthopedic surgery, neurosurgery, ophthalmological surgery, plastic and reconstructive surgery, vascular surgery, hernia surgery, head and neck surgery, hand surgery, endocrine surgery, colon and rectal surgery, breast surgery, urologic surgery, gynecological surgery, and other types of surgery. In some instances, the information about the procedures or procedure types may be more specific. For instance, the scheduling information may differentiate between an Achilles tear surgery and a meniscus repair surgery.

For each procedure, or for procedure type, the types of medical products utilized may be known or predicted. For instance, for certain types of procedures, it may be known that certain types of medical instruments, tools, disposable, or implants may be used. There may be one or more vendors that provide a particular type of medical product for a particular health care facility. For example, Hospital ABC may have multiple vendors providing various catheters. A medical personnel may have a preferred vendor. For example, a surgeon conducting an operation may have a preferred vendor that may differ from another surgeon at the same health care facility. In some instances, particular groups or departments within a health care facility may have a preferred vendor and/or type of medical product. In some instances, the preferred vendor may be noted.

The schedule may include information about which vendors be needed to provide support for a particular procedure. The schedules may or may not specify the particular vendor representative that may be needed to provide support for a particular procedure. For example the schedule may note that Surgeon X will be performing a procedure where he will need to use a catheter. It may be noted that Surgeon X always uses Catheter type A from Vendor A. A request for support for a representative from Vendor A that is knowledgeable about Catheter type A may be provided and tied in with the schedule. If no preference is indicated for a particular vendor representative, the system may automatically assign an available vendor. In some instances, a preference list may be provided that may rank the appropriate representative in the order of preference. In some instances, vendor representative availability information may be accessible as well and may be used to update the schedule. In some instances, it may be known that Surgeon X prefers working with vendor representative Smith from Vendor A (over other representatives that may be available). The request for support from Smith may be provided and tied in with the schedule.

In some instances, it may not be known whether Surgeon X has a preference for a particular product or vendor. In some instances, multiple vendors and/or vendor representatives may be put on notice. The surgeon may make a decision right before or during the procedure. The appropriate vendor representative may be notified and/or contacted at that time. The schedule may be updated in real-time to include the appropriate vendor and/or vendor representative.

A health care facility may utilize pre-existing systems and methods for managing scheduling. For example, a health care facility may have an existing software for scheduling. A communication system operated by a communication facilitation entity may communicate with the existing scheduling software and/or application programming interfaces (APIs). Alternatively, a communication system may provide scheduling software.

The scheduling information may be provided by one or more health care facilities. A communication system (such as a system operated by a communication facilitation entity), may receive the scheduling information from the one or more health care facilities. The communication system may be able to assist in formulating vendor representative schedules based on the needs of the multiple health care facilities. The communication system may be able to notify appropriate vendor representatives of a need for support based on the needs of the multiple health care facilities.

In other embodiments, the scheduling information may remain at the health care facility. The communication systems and methods may be integrated with existing health care facility infrastructure. In some instances, the health care facility may directly make requests for appropriate vendors and/or vendor representatives for support. In some instances, private information may remain within the respective health care facilities. In some instances, the scheduling information itself may not be provided to a third party and/or vendor. The desired vendor support may be requested in accordance with scheduling information that may remain within the health care facility. In some embodiments, the vendor support may be requested directly by a console at a first location (e.g., health care facility, operating room), and the requests for appropriate vendors and/or vendor representatives may be provided directly without requiring the scheduling system.

Any scheduling information may be HIPAA compliant. If some scheduling information is shared with entities outside a patient's medical care team, any personal or private information about the patient may be removed. In some instances, the information may merely include information about the vendor product and/or representative needed. Information about the procedure type may be provided. Information about the medical personnel may or may not be included.

A communication system may optionally have access to information from one or more vendors. The information may include information about vendor representatives. For example, information about vendor representative identity, vendor representative qualifications, products supported by particular vendor representatives, particular geographic regions supported by particular vendor representatives, hours that vendor representatives are typically available, vendor representative contact information, historical information about vendor representatives (health care facilities previously supported, medical personnel previously worked with), or other information may be provided by one or more vendors. Optionally, a subset or an entirety of the vendor information may be stored and/or accessible by the computer system. A communication system may optionally interface with pre-existing data management system of one or more vendors. For instance, a communication system may interface with vendor representative roster information. A communication system operated by a communication facilitation entity may communicate with the existing vendor software and/or application programming interfaces (APIs).

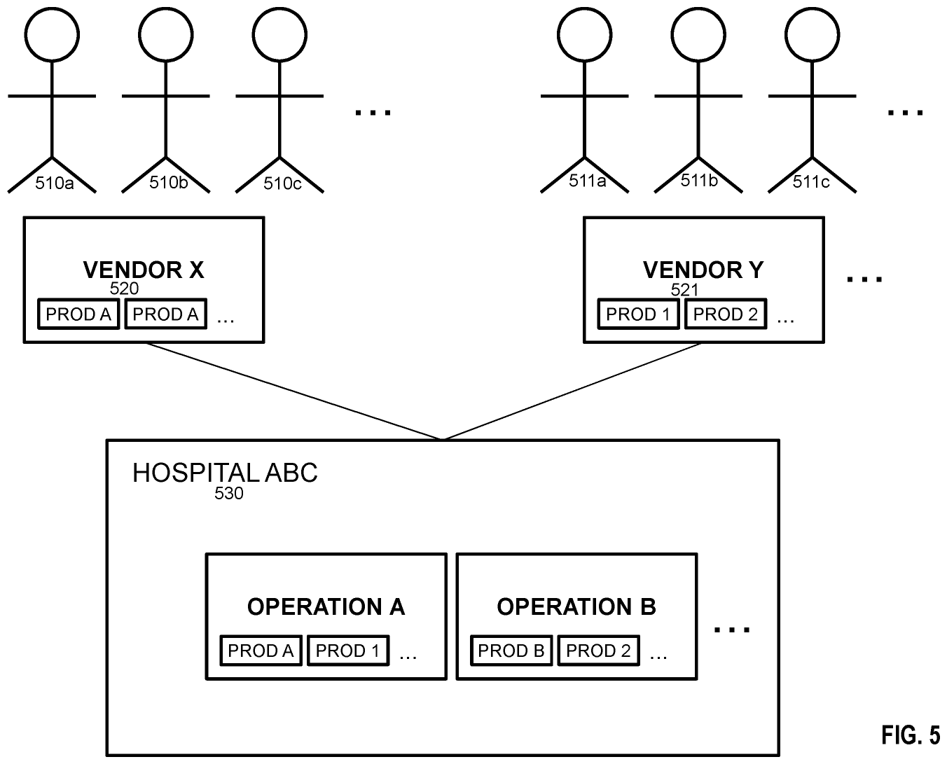

FIG. 5 shows an example of relationships between various representatives 510a, 510b, 510c, 511a, 511b, 511c, vendors 520, 521, and an exemplary health care facilities 530, in accordance with embodiments of the invention.

Any number of health care facilities may be in communication with the systems provided herein. In some embodiments, any number of health care facilities may be in communication with a central entity, or a central entity may facilitate communications between health care facilities and vendors.

A health care facility may have one or more medical procedures occurring or scheduled to occur Operation A, Operation B. A medical procedure may utilize one or more medical products PROD A, PROD B, PROD 1, PROD 2. Some medical procedures may utilize multiple medical products. The medical products may be of different types. In some embodiments, it may be predetermined which medical products will be used. In some instances, medical personnel may make a determination of which medical products to use during the medical procedure.

Various vendors may supply medical products that may be used during the procedure. For example a first vendor Vendor X may apply various products PROD A, PROD B, and a second vendor Vendor Y may supply various products PROD 1, PROD 2. Multiple vendors may supply any number of products to a respective health care facility. In some instances, different vendors may supply different types of products. In some instances, multiple vendors may supply the same type of product. For instance, multiple vendors may supply products that have clinical equivalency. For instance, a first product PROD A and a second product PROD 1 from different vendors may be used in the same situation. For example both products may be similar types of implants.

Medical personnel may have their own preferences on the exact product that they used. For example a first surgeon may prefer the first product and a second product may prefer the second product. The medical personnel may dictate which product is used during a medical procedure.

In some instances, an administrator may dictate which products are used for certain procedures. For instance, an administrator of a particular medical group or for a medical facility may dictate that a particular product be used for particular procedures. In some instances, this may change depending on the arrangements between the health care facilities and the vendors.

Each vendor may have one or more associated representatives. Vendor representatives may be associated with one or more products. For instance, a first subset of a first vendor's Vendor X representatives may be qualified to provide support for a first product PROD A while a second subset of the first vendor's representatives may be qualified to provide support for a second product PROD B. There may or may not be overlap between the first and second subsets of representatives. When a medical procedure is schedule or occurring at a health care facility, the associated vendor representatives may be put on notice. In some instances, the vendor representatives may be made aware of the procedure ahead of time. In some instances, the vendor representatives are made aware in real-time when the procedure starts. Optionally, the vendor representatives are made aware when a product that the vendor representative supports is confirmed to be used for the procedure. This confirmation may occur ahead of the medical procedure, or may occur at the start of or during the medical procedure. The confirmation may occur when a medical personnel actually starts using a medical product.

In one example, a first product PROD A or a second product PROD 1 may be used during a medical procedure Operation A. A first vendor representative 510a may provide support for the first product and a second vendor representative 511a may provide support for the second product. In some instances, both products may be used during a medical procedure. Both the first vendor representative and the second representative may be on call or may provide support as needed. In some instances, neither vendor may be needed for support. In some embodiments, the first product and the second product may be clinically equivalent and only one may be used during the procedure. The medical personnel may indicate ahead of time which product is being used so that only the associated vendor need be on call or providing support. The medical personnel may indicate in real-time as the product is being selected as used, and the associated vendor may be notified if needed for support.

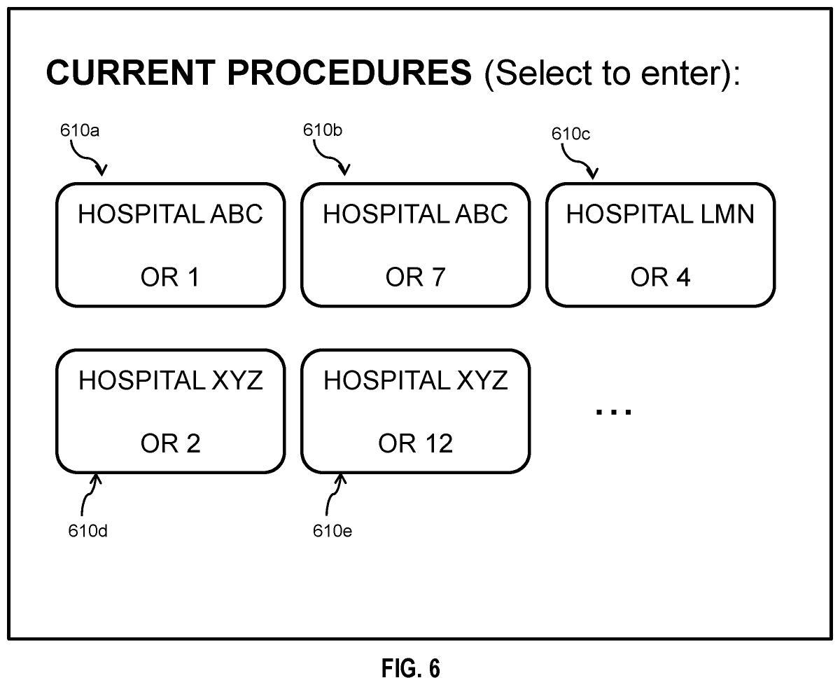

FIG. 6 shows an example of a user interface presented to a vendor representative, in accordance with embodiments of the invention. A vendor representative may provide support for one or more medical products. The one or more medical products may be associated with a particular vendor. The vendor representative may provide support to one or more health care facilities. The vendor representative may provide support remotely. The vendor representative may be able to provide support without being in the presence of the medical personnel that are using the associated medical product.

The user interface may be displayed on a rep communication device. The user interface may be shown on a screen, such as a touchscreen, of the rep communication device. A communication system, provided by a communication facilitation entity, may provide the support for the user interface displayed on the rep communication device. In some instances, the requests to the vendor representative may be based on scheduled information (e.g., may be pre-scheduled, or may be provided as a schedule is updated in real-time or periodically or in response to an event). The requests to the vendor representative may be made directly via a console at the first location (e.g., health care facility, operating room).

In one example, a vendor representative may see multiple options for procedures where the vendor representative may lend support. The user interface may present one or more visual representations of a medical procedure at a health care facility that would benefit from the vendor representative at the time that the visual representation is displayed. For example, a user interface may display multiple regions 610a, 610b, 610c, 610d, 610e which show information about various medical procedures where the vendor may lend support. The regions may include icons, images, text, or buttons. In some instances, the various regions may include information, such as the associated health care facility, the associated location at the health care facility (e.g., operating room), medical personnel (e.g., surgeon's name), type of procedure (e.g., procedure code, procedure name), medical product (e.g., identification of medical product being used). In some instances, having some information displayed in the region may give the vendor representative some context about the medical procedure to which the vendor representative will be connected. For example, if the vendor representative knows the particular product being supported, or the medical procedure that it is being used for, the vendor representative may be able to supply more relevant information in an efficient manner. If the vendor representative sees the medical personnel involved, and has worked with the medical personnel before, the vendor representative may know the personal preferences and style for the medical personnel and be able to more effectively communicate and provide support.

In some instances, the various regions may be viewable when the medical procedure is taking place or scheduled to take place. The regions may be tied into the schedule so that only current procedures are displayed. The regions may only be displayed while a need for vendor support is present. The regions may also be tied to the communication system so that if another vendor representative is providing support, the region is no longer displayed. This allows the vendor representative to view only the relevant procedures for a given time. In some instances, this may give the vendor representative an option to select a procedure to be connected to. For instance, if multiple requests are sent for support from the vendor representative, the vendor may select which procedure to be connected to.

In some instances, a visual indication of prioritization may be provided. The visual indication of prioritization may include placement of a region. For instance, the first request in time may be provided at the top or front of the list. For instance, if the system assigned the vendor representative to a particular procedure, that assignment may take precedence over later assignments to the vendor representative. In other instances, a visual indication of prioritization may include a color coding, icons, highlighting, blinking, sizing, or other displays that may draw a viewer's attention to the region. In some instances, a procedure may have higher precedence if the vendor representative's support is more urgent, or if the procedure is a higher risk procedure. The procedure may have a higher precedence if the vendor representative has previously worked with the medical personnel, or supported the same type of medical procedure at the same health care facility. The procedure may have a higher precedence if a fewer number of other vendor representatives are qualified or available to provide support.

In some instances, only a single region may be displayed on the vendor's screen. Optionally, the vendor representative's time may be reserved or dedicated to a single medical procedure at a time. The vendor may only be presented with the single relevant region for the live procedure at a given time. This may advantageously provide a simplified interface that makes it easy for the vendor to see the support request and be connected. In some instances, the vendor representative's user interface may not show the region until a live request comes in for the vendor representative's support. When the live request comes in, the vendor may see the relevant region. Optionally, the vendor representative's user interface may not show the region until the time that the medical procedure is scheduled for occurs. The region may only be viewable during a scheduled length of time for the medical procedure. The request may optionally remain visible until the vendor interacts with the region, or another vendor provides support for that instance of the medical procedure. In some embodiments, the communication system may automatically determine which medical procedure goes to which vendor representative. This may be based on one or more factors such as health care facility preference or history, group/department preference or history, individual medical personnel preference or history, availability, experience, location, user ratings for the vendor representative, associated products, or any number of factors.

The vendor representative may select a region. The representative may select a region from multiple regions displayed, or may select the only region displayed. For example, a vendor representative may touch or click on a button for a particular medical procedure. The vendor representative may be automatically connected to the location for the medical procedure (e.g., operating room). The rep communication device may automatically be put into communication with a local communication device at the location of the medical procedure. A vendor representative may be able to communicate with medical personnel via the communication devices. A vendor representative may be able to view the medical personnel, patient, and/or medical products via one or more cameras at the location of the medical procedure. One or more of the cameras may be supported by a medical console as described elsewhere herein. In some instances, one or more cameras may be built into the location, such as a ceiling, wall, or furniture of the location. This may help the vendor representative to provide medical support. Medical personnel may be able to view the vendor representative, thereby allowing the vendor representative to provide an effective telepresence.

As previously described, the vendor representative may be connected when the vendor representative selects a region. The vendor representative may be able to select the region with a single action. The single action may comprise a simple motion by the vendor representative. The vendor representative may be able to provide a single touch or single click to select the region and be automatically connected. The vendor representative may be able to provide a single swipe, or any type of single motion to be automatically connected. The vendor representative may be automatically connected when making a single interaction that does not require the vendor representative to lift the vendor's finger from the screen. The single action may optionally comprise a single motion in a single primary direction (as opposed to a complex motion with multiple primary directions). The vendor representative may be able to select the region by issuing a voice command. In some instances, the voice command may require 1, 2, 3, 4, or 5 words or syllables, or fewer. The vendor may be able to select the region by depressing a single button. The single action may require no further action or activity to create a communication channel. This may allow a vendor representative to connect to the appropriate medical procedure in a simplified manner. This facilitates communications between the vendor representative and medical personnel.

The combination of the automated presentation of medical procedures to the vendor representative on the user interface and the simple connection using a single action allows the vendor representative to more effectively communicate with associated medical personnel. This allows the vendor representative to lend support much more efficiently and effectively. The vendor representatives may benefit from being presented with information about the various medical procedures and being quickly and easily connected. The medical personnel may benefit by being presented with access to vendor representatives who may be well informed and able to focus on the support issues rather than technical details about setting up the communications. By allowing vendor representatives to remotely provide support, medical personnel may also benefit by having access to a larger pool of qualified vendor representatives.

In some embodiments, multiple vendor representatives may see a region for the same medical procedure. For example, if a particular medical product is being used for a particular procedure, there may be multiple remote vendor representatives who are qualified to provide support. In some instances, both vendor representatives may be presented with a region for that procedure. When a first vendor representative selects the region to be connected and provide medical support, that corresponding region may disappear from the second vendor representative's user interface since the first vendor representative is already connected. By allowing multiple vendor representatives to be notified, the system may allow the request for vendor representative support to be answered more quickly, since there is a larger pool of individuals notified, and there may be a greater likelihood that someone will be able to provide support.

In other embodiments, a single vendor representative may see a region for a particular medical procedure. This may optionally occur when a vendor representative is specifically requested by the medical personnel, or may somehow be preferred for that particular medical procedure. The vendor representative may select the region to be connected. If the vendor representative is unable to provide support, the vendor representative may optionally refer the medical procedure to another vendor representative. The user interface may accept an input from the vendor representative to refer the medical procedure to someone else. The system may automatically find one or more other vendor representatives who are qualified to provide support. The input from the vendor representative may be a single action or multiple action. In one example, the input may be a single action that is different from a single action to connect the vendor representative. For example, a vendor representative may tap on a region to be connected to the medical personnel. The vendor representative may swipe the region to refer the medical procedure to another vendor representative.

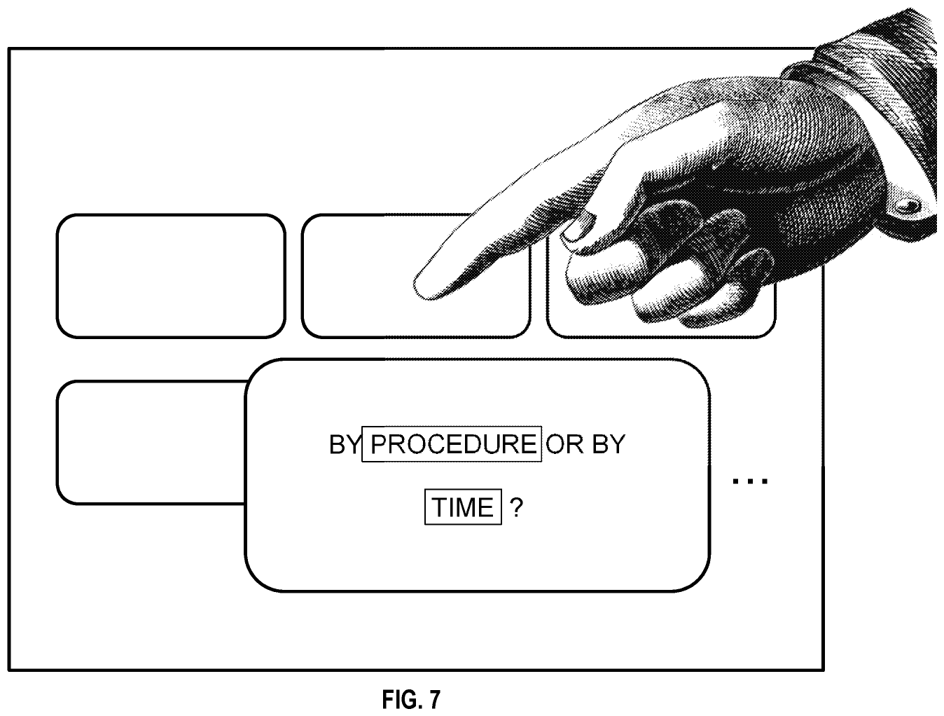

FIG. 7 shows an additional example of a user interface presented to a vendor representative, in accordance with embodiments of the invention. In some instances, the communication system may be made available to a particular vendor or vendor representative for particular periods of time or by procedure. The communication facilitation system may allow a rep communication device to communicate with a local communication device. The communication facilitation system may allow a vendor representative to communicate with medical personnel and provide support for a particular vendor product. In some instances, the system may be made available for a vendor or vendor representative for a fee. Similarly, the system may be made available for a health care facility or department/group within the health care facility for a fee.

When a user accesses the communication system, a user interface may display an option for the user to specify whether the user wishes to access the communication system by procedure or by time. The user may select an option. The user may be an administrator for a vendor. The user may be a vendor representative. In some instances, when the user selects an option, that selection may apply for the whole vendor. For example, if Vendor A has multiple representatives, a first representative selecting an option may apply for the rest of the representatives of Vendor A. Alternatively, the selection only apply to a particular user. For instance, if a first representative selects an option, a second representative may still be presented with an option for the second representative to select when the second representative accesses the system. In some embodiments, when an administrator of a vendor makes a selection of an option, the selection may apply for the various representatives.

In some embodiments, a hierarchy of users may be applied for a particular vendor. When a user that is higher up in the hierarchy makes a selection, the selection may apply to users that are lower in the hierarchy, or that operate for the particular user that makes the selection.

Optionally, the user may be an administrator for a health care facility. The user may be a medical personnel, such as a surgeon. In some instances, when the user selects an option, that selection may apply for the whole health care facility, or for the user's department/group of the health care facility. For example, if Hospital ABC has multiple surgeons, a first surgeon selecting an option may apply for the rest of the surgeons at Hospital ABC, or for the first surgeon's department or group. Alternatively, the selection only apply to a particular user. For instance, if a first surgeon selects an option, a second surgeon may still be presented with an option for the second surgeon to select when the second surgeon can accesses the system. In some embodiments, when an administrator of a health care facility makes a selection of an option, the selection may apply for the various surgeons.

In some embodiments, a hierarchy of users may be applied for a particular health care facility. When a user that is higher up in the hierarchy makes a selection, the selection may apply to users that are lower in the hierarchy, or that operate for the particular user that makes the selection.

Selecting an option to access the system by procedure may grant access for a particular procedure. For example, a vendor representative may be allowed to communicate using the communication system for a particular procedure. A flat fee may be provided for each procedure, or a fee may be determined on a number of factors, such as procedure type, procedure complexity, vendor rates, health care facility rates, and so forth. When the procedure is completed, the user may be presented with the option again to select whether to access the system by procedure or time. In other instances, the system may remember the user's earlier preference to access the system by procedure and automatically provide a per-procedure access. This per-procedure access may occur until instructed otherwise or for a predetermined period of time.

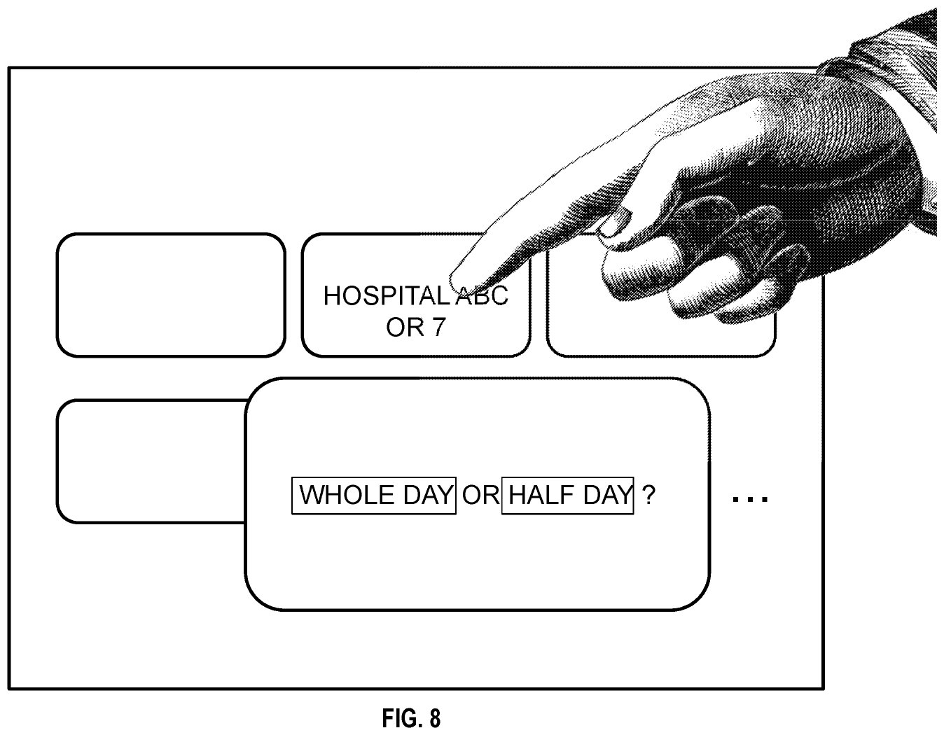

FIG. 8 shows an example of a user interface where a vendor representative may be able to establish connections with relevant personnel, in accordance with embodiments of the invention.

In some instances, a user may select an option to access the system for a period of time. For instance, the communication facilitation system may be available for a particular vendor to use to connect remotely with medical personnel and provide support. In some instances, the periods of time may be on the order of a year, a quarter, a month, several weeks, a week, several days, a day, a half day, a shift, an hour, minutes, or a minute. In some embodiments, multiple options may be presented to a user for the period of time, and the user may make a selection. For example, the user may be presented with the option to have access for a whole day or half day. Other periods of time may be presented.

In some instances, a user may enter the period of time that the user wishes to access the system. For example, the user may enter a numerical value indicative of the period of time that the user wishes to access the system. Any type of user interface may be utilized, such as buttons, drop down menus, checkboxes, forms, slider bars, or other options.

The usage information may optionally be stored by the system. The usage information may be viewable by a user of the system, such as a vendor user or health care facility user. The usage information may include information such as the procedure (or procedure type) for which the user accessed the system, and/or the length of time for which the user accessed the system. For example, Vendor A representative John Doe may have accessed the system for 72 minutes, which costs $$$ amount.

Any description herein of a vendor or vendor representative may apply to any remote user. The remote user may be authorized to have access to the information. The remote user may be a remote clinician or any other type of health care provider. The remote user may be an individual who is related to or associated with the patient. The remote user may be an individual who may support or provide additional information during a medical procedure.

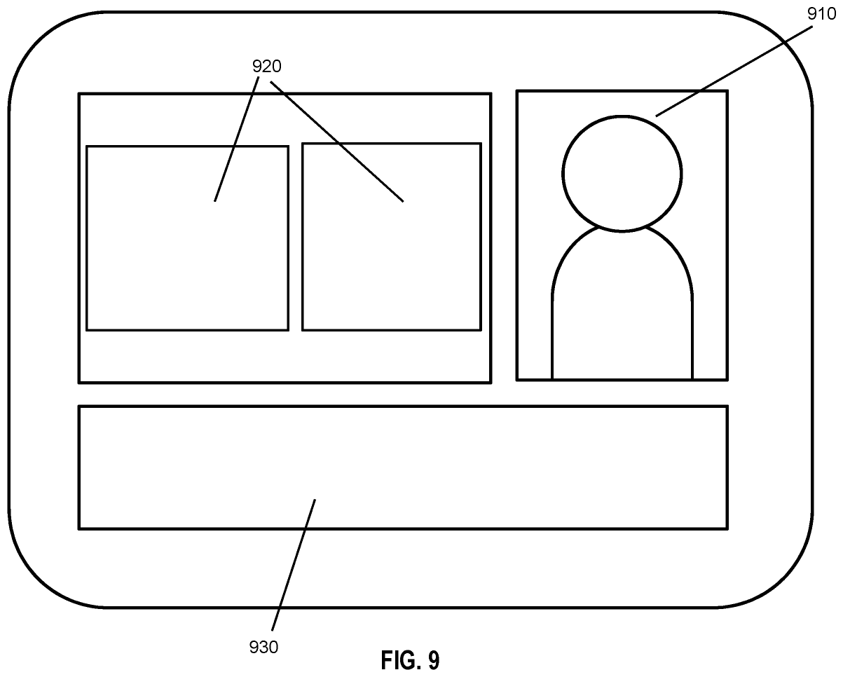

FIG. 9 shows an example of a user interface when communication has been established, in accordance with embodiments of the invention. The user interface may show any number of views. In some instances, a user interface may allow a user to view a remote individual for live chatting 910. This may allow a remote vendor representative to provide a telepresence for a medical procedure. For example, the vendor representative and a medical personnel may chat face to face.

In some embodiments, the user interface may show a surgical site and/or a product to be used 920. These views may be stable or moving as needed. The views may optionally show a surgical tool tray. In some instances, auxiliary images from devices that may be connected to be the console may be presented. For instance, images from ECG devices, endoscopes, laparoscopes, ultrasound devices, or any other devices may also be viewable. The images may be of sufficient resolution so that the medical personnel can provide effective support. The user interface may allow a vendor representative to view a relevant medical procedure and/or product and provide support as needed. The number of views and/or types of views may change as needed. A remote vendor representative may be able to control the view. For example, the remote vendor representative may be able to zoom in or out as needed. For example, a user may manipulate the images on the user's screen to zoom in or out, or to expand or reduce a particular view.

A user interface may optionally show other data 930. For example, readouts from one or more medical devices may be displayed. For example, a patient's EEG, heartrate, oxygenation level, or other data may be shown. Personal information about a patient, such as a patient's name, patient ID number, patient's demographics, patient's address, patient's medical history, may or may not be made available to view by the vendor representative.

In some instances, a vendor representative may support a single procedure or communicate with a single medical console at a time. Alternatively, a vendor representative may provide simultaneous support to multiple procedures and/or medical consoles. In some instances, a rep communication device user interface may display information relating to multiple procedures at the same time. For instance, a split screen may be employed where the type of information previously described may be displayed simultaneously (e.g., split screen, side-by-side, top-bottom viewing, etc.). In some instances, a vendor representative may be able to view information pertaining to, one, two, three, four or more procedures that the vendor representative is supporting.

In some instances, a vendor representative may be able to toggle between multiple views. For instance, if the vendor needs to provide more focused attention to a particular procedure, the vendor may zoom in or allow the information pertaining to that procedure to expand and take up more space on the screen or the entirety of the screen.

The vendor representative may optionally be able to select multiple procedures to support using the single interaction or other simplified interaction as described elsewhere herein for each of the procedures. Similarly, the vendor representative may able to select or toggle between different views using the single interaction or other simplified interaction, such as those described elsewhere herein.

### Computer Control Systems

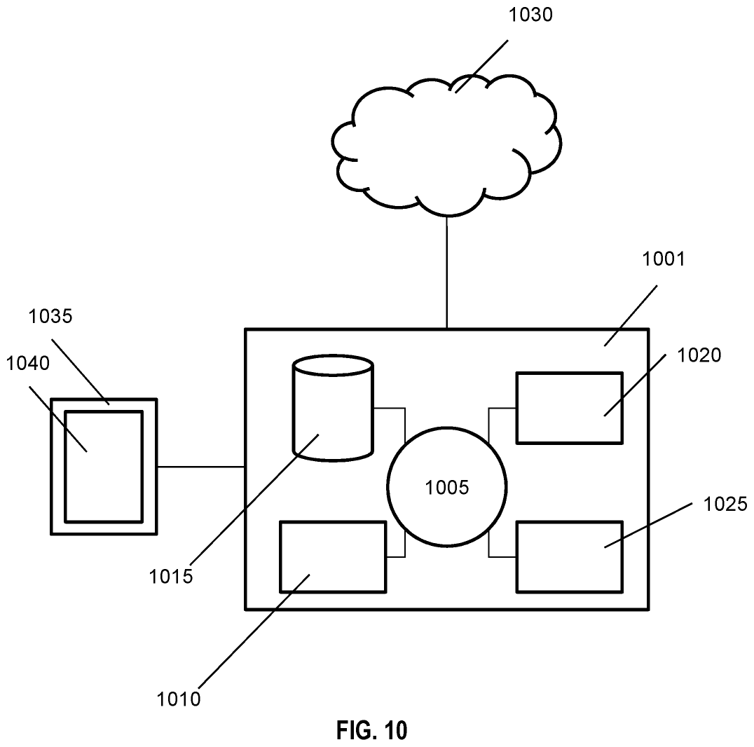

The present disclosure provides computer control systems that are programmed to implement methods of the disclosure. FIG. 10 shows a computer system 1001 that is programmed or otherwise configured to facilitate communications between vendor representatives and medical personnel that may need a vendor representative's support. The computer system may facilitate communications between a rep communication device and a local communication device. The computer system may automatically interface with one or more scheduling systems of one or more health care facilities. The computer system may access information about one or more vendors, such as one or more vendor representatives. The computer system may automatically determine one or more vendor representatives that may be suitable for providing support for a medical procedure utilizing a medical product. The computer system may optionally present one or more visual regions that allow the vendor representative to easily connect with the medical personnel. The computer system may comprise a rep communication device and/or a local communication device. The computer system may comprise, or be in communication with, one or more devices separate from the rep communication device and/or local communication device. The computer system can be an electronic device of a user or a computer system that is remotely located with respect to the electronic device. The electronic device can be a mobile electronic device.

The computer system 1001 may include a central processing unit (CPU, also “processor” and “computer processor” herein) 1005, which can be a single core or multi core processor, or a plurality of processors for parallel processing. The computer system also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet.

The network 1030 in some cases is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, capturing a configuration of one or more experimental environments; storing in a registry the experimental environments at each of one or more time points; performing one or more experimental executions which leverage experimental environments; providing outputs of experimental executions which leverage the environments; generating a plurality of linkages between the experimental environments and the experimental executions; and generating one or more execution states corresponding to the experimental environments at one or more time points. Such cloud computing may be provided by cloud computing platforms such as, for example, Amazon Web Services (AWS), Microsoft Azure, Google Cloud Platform, and IBM cloud. The network, in some cases with the aid of the computer system 1001, can implement a peer-to-peer network, which may enable devices coupled to the computer system to behave as a client or a server.

The CPU 1005 can execute a sequence of machine-readable instructions, which can be embodied in a program or software. The instructions may be stored in a memory location, such as the memory 1010. The instructions can be directed to the CPU, which can subsequently program or otherwise configure the CPU to implement methods of the present disclosure. Examples of operations performed by the CPU can include fetch, decode, execute, and writeback.

The CPU 1005 can be part of a circuit, such as an integrated circuit. One or more other components of the system can be included in the circuit. In some cases, the circuit is an application specific integrated circuit (ASIC).

The storage unit 1015 can store files, such as drivers, libraries and saved programs. The storage unit can store user data, e.g., user preferences and user programs. The computer system 1001 in some cases can include one or more additional data storage units that are external to the computer system, such as located on a remote server that is in communication with the computer system through an intranet or the Internet.

The computer system 1001 can communicate with one or more remote computer systems through the network 1030. For instance, the computer system can communicate with a remote computer system of a user (e.g., a user of an experimental environment). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, selection of an environment, a component of an environment, or a time point of an environment. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1005. The algorithm can, for example, capture a configuration of one or more experimental environments; store in a registry the experimental environments at each of one or more time points; perform one or more experimental executions which leverage experimental environments; provide outputs of experimental executions which leverage the environments; generate a plurality of linkages between the experimental environments and the experimental executions; and generate one or more execution states corresponding to the experimental environments at one or more time points.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method of performing surgery, said method comprising:
preparing one or more tools or instruments for one or more surgical procedures at a healthcare facility;
generating a visual representation of the one or more surgical procedures at the healthcare facility;
providing the visual representation to a vendor representative, wherein the vendor representative is associated with the one or more tools or instruments; and
performing one or more steps of the one or more surgical procedures based at least in part on an input provided by the vendor representative, wherein the input comprises surgical information associated with the visual representation and the one or more tools or instruments.
